(19) United States
(12) Patent Application Publication
(10) Pub. No.: US 2004/0043500 A1
(43) Pub. Date: Mar. 4, 2004

(54) Wafer-scale microwave digestion apparatus and methods

(76) Inventors: J Eric Rumps, Boise, ID (US); Ronald F. Baldner, Boise, ID (US)

Correspondence Address: TRASK BRITT, P.O. BOX 2550, SALT LAKE CITY, UT, 84110, US

(21) Appl. No.: 10/229,843

(22) Filed: Aug. 27, 2002

(51) Int. Cl. G01N 25/20; A61L 2/12

(52) U.S. Cl. 436/147; 422/99; 422/102; 422/21; 422/82.12; 422/82.13; 436/148; 436/175

(57) ABSTRACT

An apparatus and system for wafer-scale microwave digestion of layers or structures from a large-scale semiconductor substrate include a digestion vessel with a chamber which is configured to receive a whole large-scale semiconductor substrate, such as a silicon wafer. The digestion vessel may be configured to be placed within a containment apparatus, which structurally supports the digestion vessel. A digestion method includes placing at least one large-scale semiconductor substrate within the digestion vessel with a polar solvent, placing the digestion vessel within the containment apparatus, placing the containment apparatus in a microwave oven or other microwave source, and heating the polar solvent and substrate with microwaves. The heated polar solvent dissolves one or more desired layers or structures and may be subsequently analyzed to evaluate the amounts of one or more materials of such layers or structures.

### BACKGROUND OF THE INVENTION

1. Field of the Invention

The present invention relates to apparatuses for use in microwave digestion of layers or structures of semiconductor devices. More particularly, the present invention relates to apparatuses for use in effecting microwave digestion at a wafer scale. Such microwave digestion is useful in analyzing the chemistries of layers or structures of semiconductor devices and may also be useful for patterning layers or structures of semiconductor devices.

2. State of the Art

The use of microwave energy to process various kinds of materials in an efficient, economic, and effective manner to facilitate analysis of such materials is emerging as an innovative technology. Before the advent of microwave heating and microwave ovens, considerable time was required to dissolve samples for chemical analysis, especially for elemental trace analysis. Digestions were traditionally performed in open vessels on hot plates or other heating devices, resulting in long and extended digestion times which exposed laboratory personnel to caustic and harmful exhaust fumes.

Microwave heating of materials is fundamentally different from conventional radiation-conduction-convection heating. In microwave heating, the heat is generated internally within the material instead of originating from external heating sources, which creates an attractive, faster alternative to traditional heating methods and accounts for the growing popularity of microwave heating in analytical laboratories.

Another benefit of microwave heating is that it allows the rapid and direct control of heating parameters which is not available with traditional hot plate heating methods. Further, the heating efficiency of a hot plate is insufficient for many analytical processes. Hot plate heating methods are also typically performed in an open environment, which increases the risk of sample contamination. The open vessel environment traditionally associated with sample digestion using a hot plate also consumes large volumes of reagents, exposing the laboratory personnel and the laboratory to corrosive fumes.

As stated, microwave heating may be performed in either an open or closed environment. Use of a digestion apparatus or vessel allows digestion to occur under increased pressure and decreases the risk of contamination. Use of a closed vessel system provides an exponential advantage over hot plate digestion. Thus, microwave heating and digestion of samples in enclosed high pressure and high temperature vessels greatly shortens the amount of time required to perform chemical analysis.

With the advent of microwave heating and microwave ovens, elemental trace analysis has become more common, especially utilizing microwave digestion vessels. One early problem with microwave digestion was that a certain amount of guesswork was required, especially with respect to temperature, pressure, and time for a digestion procedure. Further, during microwave heating it was possible, at elevated temperatures, to cause digestion vessels to expand considerably beyond normal size. With the advent of TEFLON® (a fluoropolymer available from E. I. du Pont de Nemours & Co. of Wilmington, Del.) or other fluoropolymer molded vessels, a microwave digestion vessel was created that could function well at elevated pressures and temperatures over time.

However, closed vessel systems are not without disadvantages. A risk of explosion exists as a result of pressure build-up within the containing vessel, which could result in hazardous chemicals being emitted throughout the laboratory and obviously presents a great safety concern to laboratory personnel. The danger is enhanced, for example, if the digestion process generates gas. Thus, several closed vessel systems have been designed with pressure relief valves that vent high pressures and the collection of vapors or gases in a slow, controlled manner during microwave digestions and reduce the risk of explosion due to a pressure build-up.

In the semiconductor industry, microwave-assisted acid digestion provides a uniform and fast digestion process. As traditional microwave digestion vessels are designed for processing environmental samples, they do not accommodate an entire silicon semiconductor wafer or other large-scale fabrication substrate for examination thereof. In fact, existing vessels only permit working with a very small portion of a wafer, such as a two square inch section of a silicon semiconductor wafer. Typically, an acid, such as nitric acid or hydrochloric acid, is added to a digestion vessel containing a two-inch or smaller segment of a silicon semiconductor wafer. The small segment is then heated under pressure at a very high temperature to digest a layer or structure thereof. However, detection limits of the materials and stoichiometry of a layer or structure of a sample device are greatly enhanced when a larger sample size is used.

Thus, it will be appreciated that what is needed in the art is a microwave digestion apparatus that may be used for a wafer-scale microwave digestion of layers of structures of semiconductor devices to effect analysis thereof and for use in semiconductor device fabrication processes.

### BRIEF SUMMARY OF THE INVENTION

The present invention includes microwave digestion apparatus configured for wafer-scale microwave digestion of layers or structures of semiconductor devices to facilitate analysis of the chemistry thereof. The apparatus includes an inner sealing member, or digestion vessel, and a structurally supportive outer shell, or containment apparatus. The inner sealing member includes a base having at least one chamber configured to receive an entire fabrication substrate, or large-scale semiconductor substrate, such as a whole or partial wafer of semiconductive material (e.g., silicon, indium phosphide, gallium arsenide, etc.) or any other full or partial fabrication substrate, such as a so-called silicon-on-insulator (SOI) type substrate (e.g., silicon-on-ceramic (SOC), silicon-on-glass (SOG), silicon-on-sapphire (SOS), etc.), and a cover associated with the base such that the at least one chamber is sealed between the cover and the base. The outer shell, or containment apparatus, includes a base and a cover which, when assembled, form an enclosed chamber which is configured to receive the inner sealing member.

The inner sealing member may be formed from a chemically inert material, such as a fluoropolymer (e.g, TEFLON®). Each chamber of the inner sealing member may be defined by a sidewall extending from the base and associating with the cover. The sidewalls and cover may associate by any means that will create a seal around the chamber. For example, the cover may be secured in place relative to the sidewall by securing the cover and base of the outer shell to one another, such as by using bolts, clamps or other fasteners. In another embodiment, the cover of the outer shell may screw onto or otherwise couple with the base of the outer shell.

The inner sealing member and outer shell may both be configured to withstand a temperature of at least 300° C. Also, when assembled with one another, the inner sealing member and outer shell may be configured to withstand high pressures of approximately 500-600 psi, such as those applied within the at least one chamber as vapor is generated therein. Further, the digestion vessel and containment apparatus may comprise microwave-transparent materials.

In one embodiment, the inner sealing member includes a plurality of cavities wherein each cavity is capable of accepting an entire semiconductor substrate.

The microwave digestion apparatus may further include a temperature sensor and/or a pressure sensor that communicate with the at least one chamber of the inner sealing member. The covers of both the outer shell and the inner sealing member may include apertures configured to accommodate the temperature sensor and/or pressure sensor. In one embodiment, the temperature sensor and pressure sensor comprise a single instrument.

The present invention also includes a method of semiconductor device fabrication. The method includes creating at least one layer on a silicon wafer, sealing the whole silicon wafer within a wafer-scale microwave digestion vessel and digesting at least part of a layer on the silicon wafer. The method may further include exposing the silicon wafer to an acid prior to sealing. The acid may comprise a polar solvent such as HCl.

The present invention also includes a method for chemically analyzing layers or structures of semiconductor devices or other structures that have been fabricated on silicon wafers or other fabrication substrates. These methods include digesting the layers or structures of an entire, or whole, semiconductor substrate that are to be analyzed. Digesting may be performed in several manners. For example, digesting may include heating, under pressure, the silicon wafer or other fabrication substrate or a solvent to which at least active surfaces of the devices upon the fabrication are exposed. Digesting may further include vibrating at least the inner sealing member. The polar solvent may be an acidic solution, such as HCl or another acid. As an example of digestion in accordance with teachings of the present invention, an entire fabrication substrate may be exposed to a polar solvent while sealed within an inner sealing member and the assembly may be introduced into a microwave, which heats the polar solvent. Heating the polar solvent while it is substantially sealed within the inner sealing member may increase the pressure within the inner sealing member.

Digestion may comprise etching at least part of one layer or structure of a device or portion thereof that has been fabricated on the silicon wafer or other fabrication substrate, such as metals, metal alloys, and metal atom-containing materials, other conductive materials, electrically insulative materials, semiconductive materials, and other types of materials that may be used in the fabrication of semiconductor devices and other electronic components. By way of example only, digesting in accordance with teachings of the present invention may comprise removing an exotic metal film, such as a noble metal or noble metal alloy (i.e., rhodium and/or platinum film), from a silicon wafer.

An analysis method of the present invention may further include analyzing the elements and molecules that are present in the polar solvent following digestion. As the desired layers or structures of a whole, large-scale semiconductor substrate may be digested, a more complete picture of the layer formation process may be obtained. More specifically, the stoichiometry of the deposited material or materials may not be consistent across an entire large-scale semiconductor substrate and, as a consequence, analysis of smaller sections of the substrate, as is being conducted in the state of the art, would only provide potentially misleading information on a small portion of the area over which layer and/or structure formation processes are conducted.

The method may further include monitoring temperature and pressure conditions within the at least one chamber of the inner sealing member during the digestion process. The inner sealing member may be associated with one or both of a temperature sensor and a pressure sensor. Further, the temperature sensor and/or pressure sensor may be associated with a controller, such as a processor or smaller collection of logic circuits, which may also communicate with the microwave oven such that feedback limits control the temperature and pressure within the at least one chamber of the inner sealing member.

The method may further include at least partially enclosing the inner sealing member, or digestion vessel, within the outer shell, or containment apparatus, prior to heating the solvent. It is currently preferred that the outer shell be configured to maintain a seal provided by the inner sealing member upon an increase in pressure within the at least one chamber of the inner sealing member.

The digestion method of the present invention may also be used to etch and/or pattern material layers in semiconductor device fabrication processes.

### BRIEF DESCRIPTION OF THE SEVERAL VIEWS OF THE DRAWINGS

FIG. 1 is a cross-sectional representation of an exemplary embodiment of an inner sealing member, or digestion vessel, of a microwave digestion apparatus according to the present invention;

FIG. 2 is a cross-sectional representation of an exemplary embodiment of an outer shell, or containment apparatus, of a microwave digestion apparatus according to the present invention;

FIG. 3 is a top view of the outer shell, or containment apparatus, shown in FIG. 2;

FIG. 4 is a cross-sectional representation of an assembly including the containment apparatus of FIG. 2 and the digestion vessel of FIG. 1 being received thereby;

FIG. 5 is a cross-sectional representation of another embodiment of inner sealing member with a chamber that is configured to receive a plurality of fabrication substrates;

FIG. 6 is a cross-sectional representation of yet another embodiment of inner sealing member according to the present invention, which includes a plurality of chambers for receiving fabrication substrates;

FIG. 7 is a cross-sectional representation of a sample located within a chamber of the base of the inner sealing member, or digestion vessel, of FIG. 1 while exposed to a solvent; and

FIG. 8 is a schematic representation of use of the assembly of FIG. 6 in a microwave oven to effect digestion of one or more layers or structures that have been formed on a fabrication substrate.

### DETAILED DESCRIPTION OF THE INVENTION

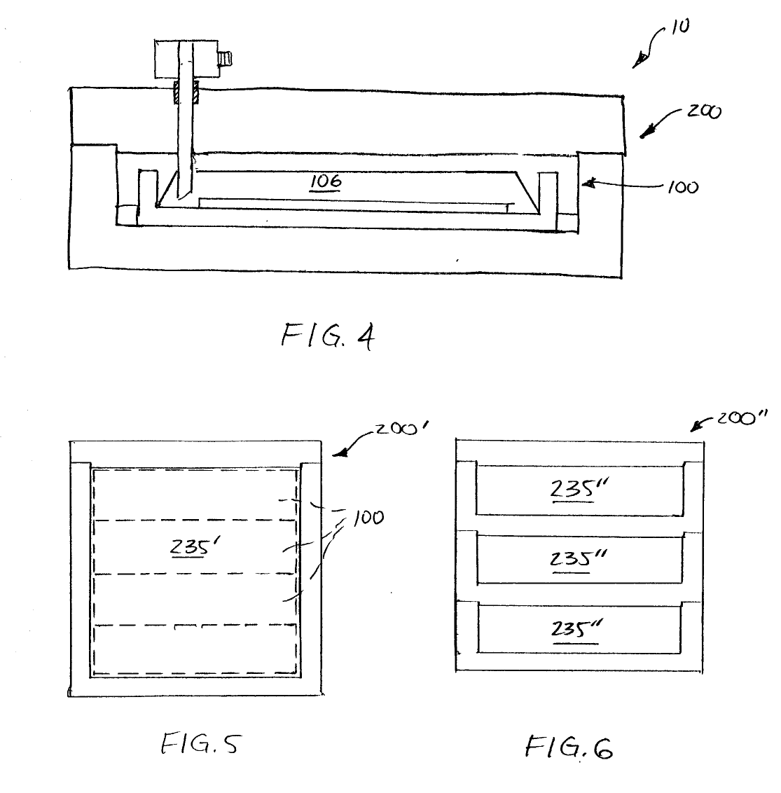

As shown in FIG. 4, the present invention includes a digestion apparatus 10 for use in a microwave oven. The digestion apparatus 10, which is also referred to herein as a microwave digestion apparatus, includes a digestion vessel 100, which is also referred to herein as an inner sealing member, and a containment apparatus 200, which is also referred to herein as an outer shell and a pressure containment apparatus. The digestion vessel 100 includes a chamber 106 configured to receive a large-scale fabrication substrate (not shown), such as a full or partial wafer of semiconductive material, an SOI-type substrate, or the like, with one or more devices (e.g., semiconductor devices) fabricated thereon. The containment apparatus 200 is configured to receive the digestion vessel 100 in such a way that a fabrication substrate within the chamber 106 thereof remains substantially sealed within the chamber 106 thereof as pressure within the chamber 106 increases or decreases.

The digestion apparatus 10 of the present invention may be used for the microwave digestion of films on a whole or partial silicon wafer or other fabrication substrate. FIG. 1 shows an exemplary embodiment of a digestion vessel 100 of a digestion apparatus 10 according to the present invention. The digestion vessel 100 includes a lower portion, or base 102, for supporting a sample 104. The sample 104 may be any desired sample to be heated under pressure. For example, the sample 104 may be a large-scale fabrication substrate, such as a whole or partial silicon wafer. In order to receive a silicon wafer that has an eight-inch circumference, the digestion vessel 100 may include a chamber 106 with a diameter of greater than about 8 inches (e.g., about 8⅛ inches).

The digestion vessel 100 also includes a cap or cover 108 which covers and substantially seals a sample 104 positioned within the chamber 106, as well as protects the sample 104 from debris and contamination. The cap 108 and base 102 of the digestion vessel 100 may fit together in any manner that creates a fluid-tight seal which will prevent material from escaping the chamber 106 of the digestion vessel 100. As shown in FIG. 1, when the cap 108 and the base 102 of the microwave digestion apparatus of the present invention are assembled, peripheral edges 114 of the cap 108 extend beyond, or overhang, the base 102 such that the cap 108 at least partially receives the base 102 and substantially encloses the chamber 106. The peripheral edges 114 may include an annular slot 116 for receiving a complementary vertical projection 118 of the lower portion 102 such that a tight seal is formed between the lower portion 102 and the cap 108. When a tight seal is formed, pressure may be increased within the digestion vessel 100.

Regulation of pressure and temperature conditions within the digestion vessel 100 is often critical to maintain desired process parameters and prevent damage to sample 104 as well as to digestion vessel 100. The digestion vessel 100 may be associated with a temperature sensor 111 and/or a pressure sensor 112 to facilitate greater control over the temperature and/or pressure within each chamber 106 of the digestion vessel 100. For example, as shown in FIG. 1, the cap 108 and lower portion 102 of the digestion vessel 100 associate to form a sealed container. Further, the digestion vessel 100 may be configured such that integration of a temperature sensor 111 and/or pressure sensor 112 does not compromise the closed, protected environment of the interior of the digestion vessel 100.

Temperature sensors 111 and/or pressure sensors 112 may provide important information for feedback to limit microwave flow once a threshold temperature and pressure are reached. The temperature sensor 111 and/or a pressure sensor 112 may be any sensor known in the art and may be automated or semiautomated. For example, a pressure sensor 112 may be associated with a valve 113 (e.g., by way of a processor 115 or smaller group of logic circuits that also communicates with the valve 113, each of which is also referred to herein as a “control element”) on the digestion vessel 100 that automatically opens (e.g., under control of the processor 115 or smaller group of logic circuits, which controls the operation of the valve 113 responsive to signals from the pressure sensor 112) when the pressure within the digestion vessel 100 exceeds a threshold value. The temperature of the solvent within the chamber 106 of the digestion vessel 100 may likewise be increased or decreased, using a microwave oven or other source of microwaves in which digestion apparatus 10 is placed, in response to a temperature signal generated by a temperature sensor 111, depending upon predetermined criteria. Alternatively, a processor 115 or other group of logic circuits that communicates with one or both of the temperature sensor 111 and the pressure sensor 112 may emit an audio or visual signal that alerts a technician that the temperature and/or pressure are approaching critical levels.

In FIG. 1, temperature sensor 111 and pressure sensor 112 are associated with or are part of a single multisensor 110. One portion of the multisensor 110 may be configured to sense temperature, while a second portion of the multisensor 110 may be configured to sense pressure. The multisensor 110 may include a single probe 120 that extends through an aperture 122 formed through the digestion vessel 100 to a location within the chamber 106 thereof which is proximate the sample 104 so as to detect pressure and or temperature conditions within the digestion vessel 100. The aperture 122 or a sealing member 123 lining at least a portion of the aperture 122 may form a substantially fluid-tight seal against the probe 120 so as to prevent fluid leakage from the chamber 106, as well as drops in the pressure within the chamber 106. Also, the aperture 122 may be reinforced, such as with a more rigid ring of material or with a resilient, compressible material secured to the regions of the digestion vessel that form the aperture 122, to compensate for the inherent weakness thereof, especially when the pressure within the chamber 106 increases.

The digestion vessel 100 is constructed of a microwave-transparent material such that the generated microwaves may pass through the digestion vessel 100 to the sample 104. The material of the digestion vessel 100 may also be intrinsically clean so that the digestion vessel 100 may be used to determine trace levels of contamination within the sample 104, as well as accurately and precisely determine the stoichiometry of the sample 104. The digestion vessel 100 may also be constructed of a material able to withstand a temperature of at least about 300° C. or greater and in such a way as to maintain very high pressure, such as the pressures that are generated as water and other polar solvents within the digestion vessel 100 begin to boil and are heated to a gaseous phase.

The material of the digestion vessel 100 may be chemically inert and not subject to corrosion by extreme conditions including high temperature and high pressure. An exemplary construction material for the interior of the digestion vessel 100 may be a fluoropolymer, such as TEFLON®, as it is substantially inert to corrosive chemicals, withstands high temperatures, and is not susceptible to fracturing or otherwise breaking.

Although FIG. 1 depicts a digestion vessel 100 which is configured to receive a single semiconductor wafer, the present invention also includes digestion vessels that may receive and enclose multiple semiconductor wafers or other large-scale substrates.

The exterior of the digestion vessel 100 may include an encasement of a material, such as KEVLAR® (a somewhat flexible, fibrous, reinforcing material available from E. I. du Pont de Nemours & Co:) or other fibrous reinforcing materials, that will provide rigidity and structural support. Alternatively, KEVLAR® or another structurally reinforcing material may be embedded within the material of the digestion vessel 100 to impart some rigidity and structural support thereto.

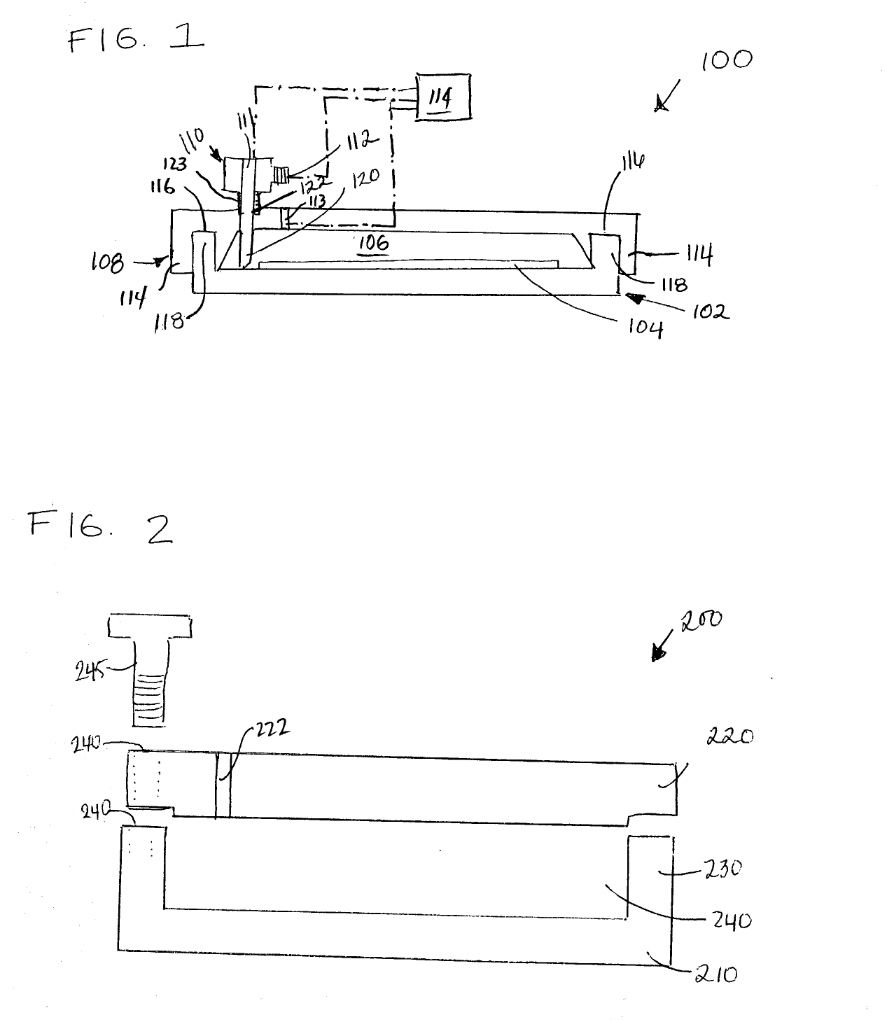

While the digestion vessel 100 may be used alone in a microwave oven or other microwave source to heat a sample under pressure, the digestion vessel 100 may alternatively be introduced into a more rigid containment apparatus 200, which maintains the shape of the digestion vessel 100 during heating thereof or of the materials therein. An exemplary containment apparatus 200 is depicted in FIG. 2.

The digestion vessel 100 and containment apparatus 200 may be constructed in such a way as to accommodate temperature and pressure sensors 111 and 112, respectively. For example, the containment apparatus 200 may be configured to accommodate the height of a multisensor 110, shown in FIG. 1. Alternatively, the containment apparatus 200 may include an opening 222 (shown in FIG. 2) through which at least a probing portion (not shown) of the temperature and/or pressure sensor 111, 112 extends.

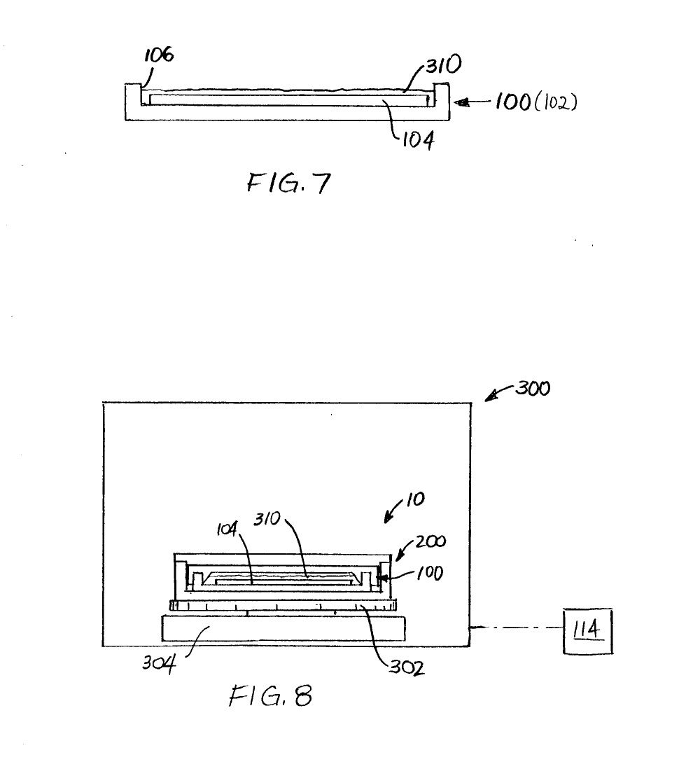

Referring to FIG. 2, the containment apparatus 200 may include a base 210 for at least partially supporting the digestion vessel 100 and a cover 220 configured to be assembled with and secured to the base 210. The base 210 may include vertical sidewalls 230 that form a chamber 235 for receiving the digestion vessel 100. Although FIG. 2 depicts a containment apparatus which is configured to receive a single digestion vessel 100, the containment apparatus 200′ of FIG. 5 may include a chamber 235′ which is configured to accommodate more than one digestion vessel 100. For example, a plurality of digestion vessels 100 may be stacked on top of each other within a single chamber 235′ of the containment apparatus 200′, as shown in FIG. 5. In another embodiment, illustrated in FIG. 6, the containment apparatus 200″ includes a plurality of chambers 235″, each of which is configured to receive and contain one digestion vessel 100 (not shown).

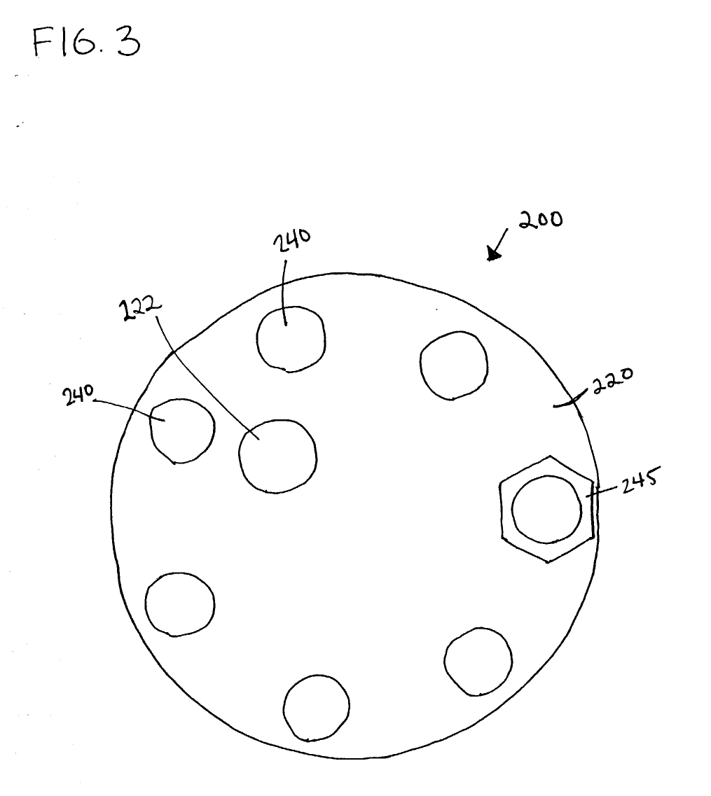

Referring again to FIG. 2, the base 210 and cover 220 of containment apparatus 200 (as well as the base and cover of containment apparatus 200′ and 200″) may fit together in any manner which creates a seal sufficient to generate and contain high temperatures and pressures. The cover 220 and base 210 may each include holes 240, 242 for receiving bolts 245. Bolt holes 242 of base 210 may be threaded so as to secure complementarily threaded bolts 245 in place upon introduction of such bolts 245 into holes 242. FIG. 3 depicts a top view of the containment apparatus 200, wherein a plurality of bolt holes 240 may receive bolts 245 for securing the cover 220 against the base (not shown in FIG. 3). Of course, containment apparatuses 200 that employ cover-securing members other than bolts, such as, for example, clamps, are also within the scope of the present invention. As shown, the cover 220 of the containment apparatus 200 also includes an opening 222 for receiving a temperature and/or pressure sensor 111 and 112, respectively.

Like the digestion vessel 100, the containment apparatus 200 may be constructed of a microwave-transparent material such that the generated microwaves may pass through the containment apparatus 200 and digestion vessel 100 to the solvent to which the sample 104 is exposed. The containment apparatus 200 may also be constructed of a material able to withstand a temperature of at least about 300° C. and in such a way as to maintain very high pressure, such as that generated as liquid materials (e.g., solvents, etchants, etc.) within the chamber 106 or cavities of the digestion vessel 100 are heated and begin to expand and/or become gaseous. An exemplary construction material for the containment apparatus 200 may be KEVLAR®, fiber-reinforced resin (e.g., fiberglass or a carbon fiber reinforced resin), steel (e.g., stainless steel), or the like. Of course, if a metal or metal alloy is used to form the containment apparatus 200, the edges and corners of the containment apparatus should be rounded or otherwise smoothed (i.e., not sharp) to avoid sparking and/or arcing when the containment apparatus 200 is exposed to microwaves.

The present invention also includes a method for digesting one or more layers and structures that have been fabricated on large-scale semiconductor substrates, such as silicon wafers. The layers or structures are formed on the large-scale semiconductor substrates as known in the art (e.g., by deposition, annealing, growth, and/or patterning processes). In analyzing the stoichiometry of layers or structures of semiconductor devices, it is often desirable to remove and/or digest at least a portion of the analyzed layers or structures. Alternatively, it may be desirable to remove all or part of a film on a large-scale semiconductor substrate during fabrication of semiconductor devices thereon. An entire large-scale semiconductor substrate, such as a whole silicon wafer, may be introduced into and sealed within a chamber of a microwave digestion vessel that incorporates teachings of the present invention to effect microwave heating of the semiconductor substrate and/or digestion chemicals, such as solvents for the layer(s) or structure(s) to be analyzed, under pressure.

By way of example only, and with reference to FIG. 7, the sample (e.g., a large-scale semiconductor substrate, such as a silicon wafer) may be exposed to a solvent 310, such as an acid, prior to sealing the sample within a digestion vessel 100 (FIG. 1). As shown in FIG. 7, the sample 104 may be placed in the chamber 106 of the base 102 of the inner sealing member 100 and exposed to a solvent 310 prior to covering the base 102 with the cover (not shown in FIG. 7). The sample 104 may be exposed to the solvent 310 prior to introduction thereof into the chamber 106 of the digestion vessel 100 or thereafter. Alternatively, the solvent 310 may be introduced into the chamber 106 of the digestion vessel 100 and the sample 104 exposed to the solvent 310 as the sample 104 is placed within the chamber 106. As an example of a solvent 310 that may be used in accordance with teachings of the present invention, digestion may be effected with a dilute acid, such as HCl. However, with microwave-assisted digestion, the increased pressure and temperature may result in the ability to achieve the same or a greater level of digestion of layers or structures with less aggressive solvents, which will result in less damage to underlying layers or structures. Any polar solvent may be used with microwave digestion. The digestion vessel 100 may then be introduced into and secured within a containment apparatus 200.

Next, as shown in FIG. 8, the assembly of the digestion vessel 100 and the containment apparatus 200 (i.e., the assembled digestion apparatus 10) may be placed within a microwave oven 300 or another microwave source of a type known in the art. During heating with the microwave oven 300, the digestion vessel 100 of the present invention may be used in combination with a turntable or other substrate-movement apparatus 302 to increase uniformity of the digestion. Similarly, the digestion vessel 100 may be vibrated, such as by use of a vibration component 304 associated with the microwave oven 300. Vibration component 304 may be configured to produce ultrasonic vibrations and/or vibrations at multiple and/or varying frequencies. Digestion typically progresses until at least a portion of a layer or structure of a sample 104 has been dissolved by the solvent 310.

The method may further include analyzing the solvent 310 and materials dissolved therein after digesting has been effected. Digesting may be performed in several manners. For example, digesting may include heating the sample 104 under pressure. For example, digesting comprises etching at least part of one layer or structure formed on a silicon wafer or other large-scale semiconductor substrate.

The method may further include monitoring temperature and pressure conditions within the chamber 106 of the digestion vessel 100 during digesting. The digestion vessel 100 may be associated with a temperature sensor 111 and/or pressure sensor 112 (see FIG. 1), described above. Further, the temperature sensor 111 and/or pressure sensor 112 may be associated with a control element, such as processor 115, that communicates with and may be used to control the operation of a microwave oven 300 such that feedback limits control the temperature and pressure within the chamber 106 of the digestion vessel 100.

Although the present invention has been shown and described with respect to various illustrated embodiments, various additions, deletions and modifications that are obvious to a person of ordinary skill in the art to which the invention pertains, even if not shown or specifically described herein, are deemed to lie within the scope of the invention as encompassed by the following claims.

What is claimed is:

1. An apparatus configured for wafer-scale microwave digestion, said apparatus comprising:
a base comprising at least one chamber, said at least one chamber having a diameter of at least about eight inches and being configured to receive a large-scale semiconductor substrate; and
a cover associated with said base such that said at least one chamber is sealed between said cover and said base.

2. The apparatus of claim 1, wherein said at least one chamber comprises a plurality of chambers.

3. The apparatus of claim 1, further comprising a temperature sensor proximate said at least one chamber.

4. The apparatus of claim 3, wherein said cover further comprises an aperture configured to accommodate said temperature sensor.

5. The apparatus of claim 1, further comprising at least one of a pressure sensor and a temperature sensor configured to sense a condition within said at least one chamber.

6. The apparatus of claim 5, wherein said cover further comprises at least one aperture configured to accommodate at least one of said pressure sensor and said temperature sensor.

7. The apparatus of claim 1, wherein said at least one chamber further comprises a fluoropolymer liner.

8. The apparatus of claim 7, wherein said base and said cover comprise a somewhat rigid material.

9. The apparatus of claim 1, wherein said base and said cover comprise a fluoropolymer.

10. The apparatus of claim 1, wherein said at least one chamber is defined by sidewalls extending from said base and associating with said cover.

11. The apparatus of claim 1, further comprising at least one digestion vessel within said at least one chamber, said at least one digestion vessel configured to enclose said large-scale semiconductor substrate.

12. The apparatus of claim 11, wherein said large-scale semiconductor substrate comprises a whole silicon wafer.

13. The apparatus of claim 1, wherein said large-scale semiconductor substrate comprises a whole silicon wafer.

14. A method for chemically analyzing at least one layer or structure formed on a large-scale semiconductor substrate, comprising:
sealing said large-scale semiconductor substrate within a chamber of a microwave digestion vessel with at least said at least one layer or structure being exposed to a solvent, said chamber having a diameter of at least about eight inches;
introducing at least said microwave digestion vessel into a microwave oven; and
heating at least one of said large-scale semiconductor substrate and said solvent with said microwave oven to effect digestion of at least part of said at least one layer or structure by said solvent.

15. The method of claim 14, wherein said sealing comprises sealing said large-scale semiconductor substrate within said microwave digestion vessel in the presence of a solvent comprising an acid.

16. The method of claim 14, wherein said sealing comprises sealing said large-scale semiconductor substrate within said microwave digestion vessel in the presence of a polar solvent.

17. The method of claim 16, wherein said sealing comprises sealing said large-scale semiconductor substrate within said microwave digestion vessel in the presence of dilute HCl.

18. The method of claim 14, further comprising analyzing at least one material dissolved within said solvent following said digestion.

19. The method of claim 14, wherein said digestion comprises heating at least one of said solvent and said large-scale semiconductor substrate under pressure.

20. The method of claim 14, wherein said digestion further comprises vibrating said microwave digestion vessel.

21. The method of claim 14, further comprising monitoring at least one of temperature and pressure conditions within said chamber during said digestion.

22. The method of claim 21, further comprising associating at least one of a temperature sensor and a pressure sensor with said microwave digestion vessel to effect said monitoring.

23. The method of claim 22, further comprising associating said temperature sensor with a microwave source and providing feedback limits to a control element associated with said microwave source.

24. A system for wafer-scale microwave digestion, comprising:
at least one digestion vessel, said at least one digestion vessel comprising at least one chamber having a diameter of at least about eight inches and being configured to receive at least one whole large-scale semiconductor substrate; and
a containment apparatus configured to receive and seal said at least one digestion vessel under pressure.

25. The system of claim 24, wherein said at least one chamber comprises a plurality of chambers.

26. The system of claim 24, wherein said at least one chamber is configured to receive a plurality of large-scale semiconductor substrates.

27. The system of claim 24, further comprising at least one of:
a temperature sensor associated with said at least one chamber; and
a pressure sensor associated with said at least one chamber.

28. The system of claim 27, wherein said containment apparatus and said at least one digestion vessel include apertures configured to accept at least a portion of said temperature sensor or said pressure sensor.

29. The system of claim 27, wherein said temperature sensor or said pressure sensor extends at least partially into said at least one digestion vessel.

30. The system of claim 24, wherein said at least one digestion vessel comprises a fluoropolymer.

31. The system of claim 24, wherein said at least one chamber of said at least one digestion vessel is configured to receive at least one whole silicon wafer.

32. The system of claim 24, wherein said at least one digestion vessel and said containment apparatus are configured to withstand a temperature of at least 300° C.

33. The system of claim 24, wherein each of said at least one digestion vessel and said containment apparatus comprises a microwave-transparent material.

34. A method of microwave-assisted digestion, comprising:
sealing a whole large-scale semiconductor substrate within a chamber of a digestion vessel, said chamber having a diameter of at least about eight inches, while exposing said whole large-scale semiconductor substrate to a polar solvent; and
heating at least said polar solvent using a microwave source.

35. The method of claim 34, wherein said exposing and said sealing comprise exposing a whole silicon wafer to said polar solvent and sealing said whole silicon wafer within said digestion vessel.

36. The method of claim 34, wherein said heating increases pressure within said digestion vessel.

37. The method of claim 36, wherein said exposing comprises exposing said whole large-scale semiconductor substrate to a polar solvent comprising an acid.

38. The method of claim 37, wherein said exposing comprises exposing said whole large-scale semiconductor substrate to dilute HCl.

39. The method of claim 34, further comprising:
analyzing said polar solvent after said heating to determine an amount of at least one material of a layer or structure dissolved by said polar solvent.

40. The method of claim 34, wherein said heating digests at least a portion of at least one layer or structure formed on said whole large-scale semiconductor substrate.

41. The method of claim 34, further comprising vibrating said digestion vessel during at least a portion of said heating.

42. The method of claim 34, wherein said heating comprises etching at least a part of a layer or structure on said whole large-scale semiconductor substrate.

43. The method of claim 34, further comprising:
associating said digestion vessel with at least one of a temperature sensor and a pressure sensor.

44. The method of claim 43, further comprising:
monitoring at least one of temperature and pressure conditions within said digestion vessel during said heating.

45. The method of claim 43, further comprising:
providing feedback limits to a control element in communication with said microwave source once at least one of a threshold temperature and a threshold pressure occurs within said digestion vessel.

46. The method of claim 34, further comprising:
sealing said digestion vessel within a containment apparatus prior to said heating, said containment apparatus configured to maintain a shape of said digestion vessel as pressure therein increases.
